(19) United States
(12) Patent Application Publication
(10) Pub. No.: US 2010/0022553 A1
(43) Pub. Date: Jan. 28, 2010

(54) Therapeutic Combination Comprising an Aurora Kinase Inhibitor and Imatinib

(75) Inventors: Jürgen Moll, Appiano Gentile (IT); Dario Ballinari, San Donato Milanese (IT); Enrico Pesenti, Parabiago (IT)

Correspondence Address: SCULLY SCOTT MURPHY & PRESSER, PC, 400 GARDEN CITY PLAZA, SUITE 300, GARDEN CITY, NY, 11530, US

(73) Assignee: NERVIANO MEDICAL SCIENCES S.R.L., Nerviano (IT)

(21) Appl. No.: 12/503,729

(22) Filed: Jul. 15, 2009

Related U.S. Application Data
(60) Provisional application No. 61/083,230, filed on Jul. 24, 2008.

(51) Int. Cl. A61K 31/496; A61K 31/506; A61P 35/00

(52) U.S. Cl. 514/252.18; 514/252.19; 514/253.06; 514/254.06

(57) ABSTRACT

The present invention provides a therapeutic combination comprising (a) a compound 1 of formula (A) as set forth in the specification and (b) a BCR-ABL kinase inhibitor selected from the group consisting of Imatinib, Dasatinib, Nilotinib, Bosutinib and Inno-406, wherein the active ingredients are present in each case in free form or in the form of a pharmaceutically acceptable salt or any hydrate thereof.

### CROSS REFERENCE TO RELATED APPLICATIONS

The present application claims benefit of U.S. Provisional Application No. 61/083,230 filed on Jul. 24, 2008.

### FIELD OF THE INVENTION

The present invention relates in general to the field of cancer treatment and, more particularly, provides an anti-tumor composition comprising an Aurora kinase inhibitor and a BCR/ABL kinase inhibitor having a synergistic or additive antineoplastic effect

### BACKGROUND OF THE INVENTION

Survival rates in Chronic Myclogenous Leukemia patients have improved dramatically since the introduction of Imatinib (Glivec, Gleevec) in 2001, a tyrosine kinase inhibitor, that is highly effective against most cases of CML in chronic phase, but remains poorly active in patients in the blast phase. Imatinib targets BCR-ABL, which is the major cause of CML and a subset of ALL patients bearing the Philadelphia chromosome. For review see: Deininger M, Buchdunger E, Druker B J. The development of Imatinib as a therapeutic agent for chronic myeloid leukemia. Blood 2005;105:2640-53.

In particular patients in advanced phases of CML are resistant a priori or frequently develop resistance to Imatinib therapy, which is often due to the emergence of mutant forms of Bcr-Abl bearing point mutations in the kinase domain. These mutations interfere either directly with binding of the drug or prevent the adoption of the inactive conformation required for binding. Since Dasatinib and Nilotinib have been launched most of the mutations have a treatment option, with the exception of one of the most common identified mutations, which is located in the gatekeeper residue Threonine 315 of Abl and which is mutated towards an Isoleucine (T315I). Against this mutation the most advanced second generation BCR-ABL inhibitors such as Dasatinib, Nilotinib, Bosutinib or Inno-406 are inactive.

Compound 1 has been identified based on a biochemical screen for inhibitors of Aurora kinases and shows cross-reactivity with Abl kinase (see P. Carpinelli et al., Mol Cancer Ther 6: 3158-3168.)

The Aurora kinase inhibitor Compound 1 was also tested preclinically for its activity to inhibit proliferation of cell lines expressing wildtype or Imatinib resistant BCR-ABL mutants including the T315I mutant and its crystal structure in complex with T315I Abl mutant has been solved (see Modugno et al., Crystal structure of the T315I Abl mutant in complex with the aurora kinases inhibitor Compound 1. Cancer Res. Sep. 1, 2007;67(17):7987-90). In these cells both Abl and Aurora kinase activity were inhibited and Compound 1 showed pharmacological synergy with Imatinib in cell lines with a partial resistance to Imatinib. Strong antiproliferative activity is also seen in CD34+ cells from CML patients in chronic phase or blast crisis, including those bearing the T315I mutation (Gontarewicz, A. et al. Simultaneous targeting of Aurora kinases and Bcr-Abl kinase by the small molecule inhibitor Compound 1 is effective against Imatinib-resistant BCR-ABL mutations including T315I Blood (2008) vol. 111, p. 4355-4364).

There is a continuous need of combination of known anticancer drugs in order to optimise the therapeutic treatment.

Some pyrrolopyrazoles have been demonstrated to be potent inhibitors of Aurora kinase enzymes. One of these compounds is currently in development as an anti-cancer agent. Aurora kinase inhibitors are understood to trigger an aberrant mitosis, dependent on the genetic background of cells leading to a G2/M block, endoreduplication and/or apoptosis.

The present invention provides new combinations of a kinase inhibitor, targeting Aurora kinases as well as wild-type and mutant ABL kinase, with known pharmaceutical agents that are particularly suitable for the treatment of proliferative disorders, especially CML. More specifically, the combinations of the present invention are very useful in therapy as antitumor agents and lack, in terms of both toxicity and side effects, the drawbacks associated with currently available antitumor drugs.

### SUMMARY OF THE INVENTION

The present invention provides a therapeutic combination comprising (a) Compound 1 of formula (A):

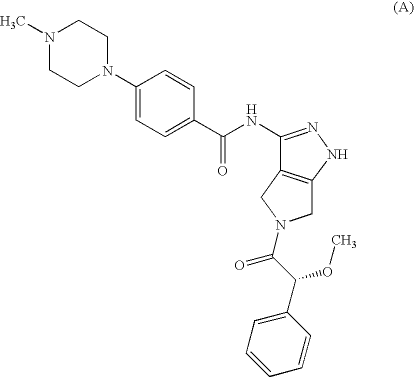

and (b) a BCR-ABL kinase inhibitor, wherein the active ingredients are present in each case in free form or in the form of a pharmaceutically acceptable salt or any hydrate thereof.

The present invention also provides a method of treating or delaying the progression of a proliferative disorder, wherein said method comprises the simultaneous, sequential or separate administration to a patient in need thereof of the above-mentioned therapeutic combination.

The present invention further provides a pharmaceutical composition comprising the above-identified therapeutic combination admixed with a pharmaceutically acceptable carrier, diluent or excipient.

### DETAILED DESCRIPTION OF THE INVENTION

The present invention provides, in a first embodiment, a therapeutic combination comprising (a) Compound 1 of formula (A):

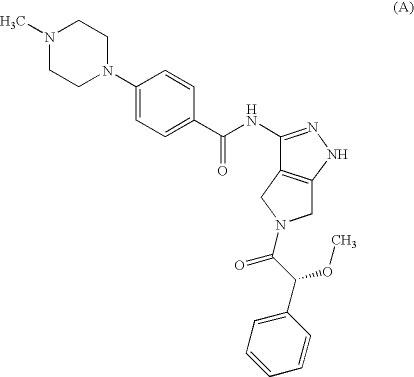

and (b) a BCR-ABL kinase inhibitor, wherein the active ingredients are present in each case in free form or in the form of a pharmaceutically acceptable salt or any hydrate thereof

A further embodiment of the combination according to the invention is a combined preparation for simultaneous, separate or sequential use.

A still further embodiment relates to the combination according to the invention in a method of treating or delaying the progression of a proliferative disorder, wherein the method comprises the simultaneous, sequential or separate administration to a patient in need thereof of the therapeutic combination.

In a still further embodiment the invention provides a pharmaceutical composition comprising a combination according to the invention admixed with a pharmaceutically acceptable carrier, diluent or excipient.

Another embodiment relates to the use of a compound 1 of formula (A) as defined above in the preparation of a medicament for the treatment of a proliferative disorder, wherein said treatment comprises simultaneously, sequentially or separately administering a compound of formula (A) as defined above and a BCR-ABL kinase inhibitor selected from the group consisting of Imatinib, Dasatinib, Nilotinib, Bosutinib and Inno-406, to a patient in need thereof.

Still another embodiment relates to the use of a compound of formula (A) as defined above and a BCR-ABL kinase inhibitor, in the preparation of a medicament for treating a proliferative disorder.

The compound 1 of formula (A) has the chemical name N-[5-(2-Methoxy-2-phenyl-acetyl)-1,4,5,6-tetrahydro-pyrrolo[3,4-c]pyrazol-3-yl]-4-(4-methyl-piperazin-1yl)-benzamide. This compound was described and claimed in the international patent application WO2005/005427, published on Dec. 20, 2005, which also disclosed the process for its preparation (incorporated herein by reference). The compound 1 of formula (A) is endowed with protein kinase inhibitory activity and is thus useful in therapy as an antitumor agent.

Pharmaceutically acceptable salts of the compound 1 of formula (A) include the acid addition salts with inorganic or organic acids, e.g., nitric, hydrochloric, hydrobromic, sulphuric, perchloric, phosphoric, acetic, trifluoroacetic, propionic, glycolic, lactic, oxalic, malonic, malic, maleic, mesylate, tartaric, citric, benzoic, cinnamic, mandelic, methanesulphonic, isethionic and salicylic acid and the like.

According to a preferred embodiment of the invention, the BCR-ABL inhibitors are selected from the group consisting of Imatinib, Dasatinib, Nilotinib, Bosutinib and Inno-406. In a more preferred embodiment of the invention, the BCR-ABL inhibitor is Imatinib.

Imatinib can be administered, e.g., in the form as it is marketed, e.g. under the trademark Glivec® or Gleevec®. Dasatinib can be administered, e.g., in the form as it is marketed, e.g. under the trademark Sprycel®. Nilotinib can be administered, e.g., in the form as it is marketed, e.g. under the trademark Tasigna®.

In the present invention, each of the active ingredients of the combination is in an amount effective to produce a synergistic or additive antineoplastic effect.

The present invention also provides a method for lowering the side effects caused by antineoplastic therapy with an antineoplastic agent in mammals, including humans, in need thereof, the method comprises administering to said mammal a combined preparation comprising the compound 1 of formula (A) as defined above and a BCR-ABL inhibitor selected from the group consisting of Imatinib, Dasatinib, Nilotinib, Bosutinib and Inno-406, in amounts effective to produce a synergistic or additive antineoplastic effect.

By the term “a synergistic antineoplastic effect” as used herein is meant the inhibition of the growth of the tumor, preferably the complete regression of the tumor, by administering an effective amount of the combination of a the compound of formula (A) as defined above and a BCR-ABL inhibitor selected from the group consisting of Imatinib, Dasatinib, Nilotinib, Bosutinib and Inno-406 to mammals, including humans.

The term “combined preparation” as used herein defines especially a “kit of parts” in the sense that the combination of components (a) and (b) as defined above can be dosed independently or by use of different fixed combinations with distinguished amounts of the combination components (a) and (b), i.e. simultaneously or at different time points. The elements of the kit of parts can then, e.g., be administered simultaneously or chronologically staggered, that is at different time points and with equal or different time intervals for any part of the kit of parts. More preferably, the time intervals are chosen such that the effect on the treated disease in the combined use of the parts is greater than the effect which would be obtained by use of only any one of the combination components (a) and (b). The ratio of the total amounts of the combination component (a) to the combination component (1) to be administered in the combined preparation can be varied, e.g. in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination components (a) and (b), in particular a synergism, e.g. a more than additive effect, additional advantageous effects, less side effects, less toxicity, and more preferably a strong synergism of the combination components (a) and (b). In addition, a beneficial effect is a combined therapeutic effect in a dosage where component (a) and/or component (b) has no therapeutic effect alone under such dosage.

By the term “administered” or “administering” as used herein is meant parenteral and/or oral administration. By “parenteral” is meant intravenous, subcutaneous and intramuscular administration.

In the method of the subject invention, for the administration of the compound 1 of formula (A), the course of therapy generally employed is in the range from 100 mg/m²/day to 1500 mg/m²/day of body surface area for up to 21 consecutive days. More preferably, the course therapy employed is from about 150 mg/m²/day to about 350 mg/m²/day of body surface area for up to 21 consecutive days. In a particularly preferred regimen, the compound of formula (A) is administered in a dose of 250, 330, or 400 mg/m²/day of body surface area for six hours infusion on days 1, 8 and 15 of a four weeks cycle. Other possible therapeutic schedules are disclosed, for example, in WO 2008/052931 published May 8, 2008 (incorporated herein by reference).

The compound 1 of formula (A) can be administered in a variety of dosage forms, e.g., orally, in the form of tablets, capsules, sugar or film coated tablets, liquid solutions or suspensions; rectally in the form of suppositories; parenterally, e.g., intramuscularly, or through intravenous and/or intrathecal and/or intraspinal injection or infusion.

For the administration of a BCR-ABL inhibitor the course of therapy generally employed for Imatinib is from 150 mg/m²/day to 700 mg/m²/day, more preferably, from about 200 mg/m²/day to 350 mg/m²/day. For Dasatinib, the dose regimen is about 70 mg per os bid.

The antineoplastic therapy of the present invention is in particular suitable for treating gastro-intestinal tumour (GIST) or hematopoietic malignant tumours such as leukaemias and lymphoma (i.e. Acute Lymphoblastic Leukaemia (ALL), Chronic Lymphocytic Leukaemia (CLL), Multiple Myeloma (MM), Chronic Mycloid Leukaemia (CML), Acute Myeloid Leukaemia (AML)).

As stated above, the effect of the combination of the invention is significantly increased without a parallel increased toxicity. In other words, the combined therapy of the present invention enhances the antitumoral effects of the component (a) and/or of component (b) of the combination of the invention and thus yields the most effective and less toxic treatment for tumors.

Pharmaceutical compositions according to the invention are useful in anticancer therapy.

The present invention further provides a commercial package comprising, in a suitable container means, (a) a compound 1 of formula (A) as defined above, and (b) a BCR-ABL inhibitor, wherein the active ingredients are present in each case in free form or in the form of a pharmaceutically acceptable salt or any hydrate thereof, together with instructions for simultaneous, separate or sequential use thereof.

In a package according to the invention each of components (a) and (b) are present within a single container means or within distinct container means.

Another embodiment of the present invention is a commercial package comprising a pharmaceutical composition or product as described above.

Due to the key role of the Aurora kinases in the regulation of celular proliferation, the combinations of the present invention are also useful in the treatment of a variety of cell proliferative disorders such as, for example, benign prostate hyperplasia, familial adenomatosis, polyposis, neurofibromatosis, psoriasis, vascular smooth cell proliferation associated with atherosclerosis, pulmonary fibrosis, arthritis, glomerulonephritis and post-surgical stenosis and restenosis.

The activities of the combination of the present invention are shown for instance by the following in vitro and in vivo tests, which are intended to illustrate but not to limit the present invention.

The synergistic antineoplastic effect of the combined preparations of the present invention is shown, for instance, by the following in vitro test, which is intended to illustrate the present invention without posing any limitation to it.

### Example 1

### In Vitro Anti-Proliferative Effect of Compound 1 in Combination with Imatinib

Table 1 reports the results obtained testing in vitro the cytotoxic effect of Compound 1 in combination with Inatinib.

Materials and Methods: Exponentially growing human myelogenous leukemia K-562 cell line was seeded and incubated at 37° C. in a humidified 5% CO₂ atmosphere. Drugs were added to the experimental culture, and incubations were carried out at 37° C. for 72 hours in the dark. Scalar doses of Compound 1 and Imatinib were added to the medium 24 hours after seeding.

Three treatment schedules were tested: A) simultaneous administration (both drugs administered to cells for 72 hours); B) sequential administration (Compound 1 administered 24 hours before Imatinib). C) sequential administration (Imatinib administered 24 hours before Compound 1).

Drug solutions were prepared immediately before use. At the end of treatment, cell proliferation was determined by counting the cell number using a Coulter Counter.

Inhibitory activity was evaluated comparing treated versus control data using Assay Explorer (MDL) program. The dose inhibiting 50% of cell growth was calculated using sigmoidal interpolation curve. Combination indices (C.I.) were calculated using a computer program for multiple drug effect analysis based on the equation of Chou-Talalay (Adv Enzyme Regul 1984; 22:27-55) for mutually nonexclusive drugs, where a C.I.<1 indicates a more than additive effect (C.I.>3 indicates strong antagonism; 1.3<C.I.<3, antagonism; 0.8<C.I.<1.2, additivity; 0.3<C.I.<0.8, synergism; C.I.<0.3, strong synergism).

Results. The administration to human myelogenous leukemia K-562 cell lines of Compound 1 in combination with Imatinib resulted in a synergistic antitumor effect.

TABLE 1

| | | Drug | C.I. at 70% of | Effect of |
|---|---|---|---|---|
| Cell Line | Schedule | RATIO | fraction affected | Combination |
| K-562 | A | 1:0.5 | 0.24 | strong synergism |
| | | 1:1 | 0.21 | strong synergism |
| | | 1:2 | 0.51 | synergism |
| | | 1:4 | 0.49 | synergism |
| | B | 1:0.05 | 0.06 | strong synergism |
| | | 1:0.1 | 0.01 | strong synergism |
| | | 1:0.2 | 0.19 | strong synergism |
| | | 1:0.4 | 0.09 | strong synergism |
| | C | 1:0.05 | 0.22 | strong synergism |
| | | 1:0.1 | 0.30 | synergism |
| | | 1:0.2 | 0.74 | synergism |
| | | 1:0.4 | 0.55 | synergism |

### Example 2

### In vivo antitumor efficacy in combination with Imatinib

SCID female mice, from Harlan (Italy), were maintained in cages with paper filter cover, food and bedding sterilized and water acidified. Human myeloid leukemia K-562 cell line was maintained in vitro at 37° C. in a humidified 5% CO₂ atmosphere.

For in vivo experiments 10⁷ K562 cells were implanted subcutaneously in SCID mice. K562 cell line was selected as it is a BCR-ABL positive model carrying the chromosomal translocation known as Philadelphia chromosome and because it was previously demonstrated that it is sensitive to Imatinib.

On day 7, when tumors reached an estimated weight of 100 to 150 mg, animals were assigned to 4 experimental groups by random selection and received the following treatments: group 1, control, vehicle solution; group 2, Compound 1 twice a day intraperitoneally at a dose of 15 mg/kg for 9 consecutive days (days 7, 8, 9, 10, 11, 12, 13, 14, 15); group 3, Imatinib twice a day per os at 100 mg/kg for 9 consecutive days (days 7, 8, 9, 10, 11, 12, 13, 14, 15); and group 4 Compound 1 twice a day intraperitoneally at a dose of 15 mg/kg (days 7, 8, 9, 10, 11, 12, 13, 14, 15) and Imatinib twice a day per os at 100 mg/kg (days 7, 8, 9, 10, 11, 12, 13, 14, 15).

Tumor growth and body weight were measured every 3 days. Tumor growth was assessed by caliper. The two diameters were recorded and the tumor weight was calculated according the following formula: length (mm)×width²/2 The effect of the antitumor treatment was evaluated as the delay in the onset of an exponential growth of the tumor (see for references, Anticancer drugs 7:437-60,1996). This delay (T-C value) was defined as the difference of time (in days) required for the treatment group (T) and the control group(C) tumors to reach a predetermined size (1 g).

Toxicity was evaluated on the basis of body weight reduction. The results are reported in Table 2 below. Compound 1 combined with Imatinib produced a clear therapeutic advantage: the T-C observed when Compound 1 was combined with Imatinib was clearly superior to the one obtained with Imatinib or Compound 1 as single agent. No toxicity was observed in any of the treatment group.

TABLE 2

| | Time to reach | | |
|---|---|---|---|
| Treatment | 1 g (days) | T-C (days) | Toxicity |
| Compound 1 | 27.3 | 11.5 | 0/7 |
| 15 mg/kg* | | | |
| Imatinib | 25.1 | 9.3 | 0/7 |
| 100 mg/kg** | | | |
| Imatinib | 35.9 | 20 | 0/7 |
| 100 mg/kg + | | | |
| Compound 1 | | | |
| 15 mg/kg*** | | | |

*Treatments made intraperitoneally twice a day on days 7, 8, 9, 10, 11, 12, 13, 14, 15
**Treatments made per os at days 7, 8, 9, 10, 11, 12, 13, 14, 15
***Days 7, 8, 9, 10, 11, 12, 13, 14, 15 Imatinib treatments; days 7, 8, 9, 11, 12, 13, 15 Compound 1 treatments.

1. A therapeutic combination comprising (a) Compound 1 of formula (A): 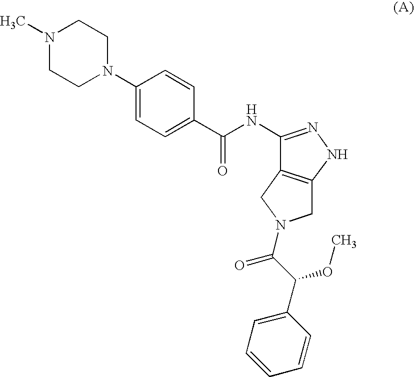
and (b) a BCR-ABL kinase inhibitor, wherein the active ingredients of the combination are present in free form or in the form of a pharmaceutically acceptable salt or any hydrate thereof.

2. The combination according to claim 1 wherein the BCR-ABL kinase inhibitor is selected from the group consisting of Imatinib, Dasatinib, Nilotinib, Bosutinib and Inno-406.

3. The combination according to claim 2 wherein the BCR-ABL kinase inhibitor is Imatinib.

4. A method of treating or delaying the progression of a proliferative disorder comprising the simultaneous, sequential or separate administration to a patient in need thereof of a therapeutically effective amount of the combination according to any one of claims 1 to 3.

5. A pharmaceutical composition comprising a combination according to any one of claims 1 to 3 admixed with a pharmaceutically acceptable carrier, diluent or excipient.

6. A method for lowering the side effects caused by antineoplastic therapy with an antineoplastic agent in humans in need thereof comprising the simultaneous, sequential or separate administration to said humans the combination according to any one of claims 1 to 3, in amounts effective to produce a synergistic antineoplastic effect.

7. A commercial package comprising, in a suitable container means, the combination according to any of claims 1 to 3, together with instructions for simultaneous, separate or sequential use thereof.
